(19) United States
(12) Patent Application Publication
(10) Pub. No.: US 2011/0093014 A1
(43) Pub. Date: Apr. 21, 2011

(54) Rod with Removable End and Inserter Therefor

(75) Inventors: Reginald James Davis, Cockeysville, MD (US); Peter Thomas Miller, Austin, TX (US); Charles R. Forton, Leander, TX (US)

(73) Assignee: Zimmer Spine, Inc., Minneapolis, MN (US)

(21) Appl. No.: 12/581,705

(22) Filed: Oct. 19, 2009

(51) Int. Cl. A61B 17/70; A61B 17/88

(52) U.S. Cl. 606/259; 606/279

(57) ABSTRACT

Embodiments disclosed herein provide a rod and a corresponding rod inserter. The rod has an elongated body with a removable end configured for an insertion tool engagement. The removable end of the rod has a part that can be securely attached to a rod holder of a rod inserter ex situ prior to minimally invasive rod insertion. A breakable portion is located between the part and the elongated body. The part can be broken off from the rod in situ to release the rod inserter. The broken off part of the rod is removed with the rod inserter and separated from the rod inserter ex situ.

### TECHNICAL FIELD OF THE DISCLOSURE

Embodiments of the disclosure relate generally to spinal stabilization implants and instruments. More particularly, embodiments of the disclosure relate to a rod with a removable end and an inserter for inserting the rod and shearing off the removable end of the rod in a minimally invasive manner.

### BACKGROUND OF THE RELATED ART

The spine is subject to abnormal curvature, injury, infections, tumor formation, arthritic disorders, and puncture or slippage of the cartilage disks. Modern spine surgery often involves the use of spinal implants to help stabilize the spine, correct deformities of the spine, facilitate fusion, or treat spinal fractures. Some spinal implants such as a spinal stablization system may provide support for the affected regions of the spine.

Often, spinal stabilization systems include rods which can bear a portion of the forces that would otherwise be transmitted along the spine. These rods may be implanted in pairs or in other numbers along portions of the spine of interest. Some spinal stabilization systems may support a portion of the spine including only two vertebrae (and associated anatomical structures) while some spinal stabilization systems support multiple levels of vertebrae. Spinal stabilizations systems can be used to support various portions of the spine, including the lumbar portion of the spine and the thoracic portion of the spine. Regardless of the number of rods implanted, or the portion of the spine in which they may be implanted, the rods can be attached to one or more vertebrae of the spine to provide support and stabilize, align, or otherwise treat the region of the spine of interest. Surgical personnel may use one or more anchor systems to attach the rods to one or more vertebrae. One such anchor system includes pedicle screws constructs which define slots, keyways, grooves, apertures, or other features for accepting and retaining stabilization rods which may be static, dynamic, or a combination of both. In many pedicle screw constructs, pedicle screws are placed in vertebrae selected by surgical personnel.

During some surgical procedures, a rod may be inserted into a patient or otherwise delivered to a work site in a minimally invasive manner (less than an open procedure), such as percutaneously. Percutaneous rod insertion in a minimally invasive surgery (MIS) generally requires an insertion instrument with a strong grip on the rod to prevent loosening or toggling of the rod. This allows the rod to be properly steered into the pedicle screws for positioning and locking.

Often, these inserters rely on a mechanism that is in situ and that is manually locked using other tools or a feature integral to the insertion instrument. For example, in addition to an insertion instrument specifically designed to lock onto or otherwise securely attach itself to a rod for percutaneous rod insertion, another instrument may be needed to unlock or otherwise detach the insertion instrument from the rod so the insertion instrument can be safely retrieved after the rod is properly inserted and positioned in the pedicle screws. Some inserters may have a built-in feature integral to the instrument that can be used to unlock and/or detach the insertion instrument from the rod.

### SUMMARY OF THE DISCLOSURE

Prior rod inserters generally rely on a mechanism that is in situ. Because it can be difficult to insert mating instrumentation and find the unlocking mechanism in situ, an ex situ solution is preferable. Embodiments disclosed herein provide a rod with a removable end and a corresponding rod inserter. The removable end of the rod can be securely attached to a rod holder at the distal end of the rod inserter ex situ. Using the rod inserter, the rod may be inserted percutaneously in a minimally invasive manner. The removable end of the rod can then be broken off or otherwise removed to release the rod inserter.

To provide a surgeon with a strong grip on the rod to prevent loosening or toggling of the rod during the insertion, in some embodiments, a pin may be inserted through a hollow body of the rod inserter and into a cavity or hole at the removable end of the rod. The interior of the hole at the removable end of the rod and a portion of the exterior of the pin at the distal end may be threaded to mate with each other. In some embodiments, the hole at the removable end of the rod may be female threaded to mate with a male threaded pin or screw. For example, the rod holder of the inserter may have an opening through which a set screw may be inserted to mate with the cavity or hole at the removable end of the rod. Fastening the pin or screw onto the removable end of the rod through the rod inserter effectively locks the distal end of the rod inserter with the removable end of the rod. This locking mechanism can only be locked and unlocked ex situ. More specifically, after the rod is properly inserted and the removable end broken off from the rod, the rod inserter is released from the rod and retrieved with the broken off portion of the rod securely held inside the rod holder via the pin or screw. The broken off portion of the rod can be separated from the rod inserter by unscrewing the pin or screw ex situ.

In some embodiments, the rod may have a structurally weakened point or portion at the removable end of the rod to allow a portion thereof to break away from the rest of the rod with the use of force. This may be a counter-torque force applied onto pedicle screws to fully tighten them to the rod.

In addition to the removable end, in some embodiments, a rod may have a blunt and/or tapered end. In some embodiments, the rod may be sized and/or dimensioned to suit. Those skilled in the art will appreciate that either end or both ends of the rod may also be sized and/or dimensioned to suit.

Embodiments disclosed herein can provide many advantages. For example, no additional instrumentation is required to unlock the inserter in situ. By eliminating the need for in situ unlocking mechanism, embodiments disclosed herein may advantageously and desirably enhance the minimally invasive surgical technique. Moreover, embodiments disclosed herein can be used in longer constructs that cannot otherwise be performed using prior rod inserters.

Other objects and advantages of the embodiments disclosed herein will be better appreciated and understood when considered in conjunction with the following description and the accompanying drawings.

### BRIEF DESCRIPTION OF THE DRAWINGS

A more complete understanding of the present invention and the advantages thereof may be acquired by referring to the following description, taken in conjunction with the accompanying drawings in which like reference numbers indicate like features and wherein:

FIG. 1A depicts a diagrammatic representation of a perspective view of an example embodiment of a rod having a removable end;

FIG. 1B depicts a diagrammatic representation of a side view of the rod shown in FIG. 1A;

FIG. 2A depicts a diagrammatic representation of a perspective view of the rod shown in FIG. 1A after a portion of the rod is broken off or otherwise detached from the rod;

FIG. 2B depicts a diagrammatic representation of a side view of the rod shown in FIG. 2A;

FIGS. 3A and 3B depict diagrammatic representations of side views of an example embodiment of a rod inserter;

FIG. 3C depicts a diagrammatic representation of a close-up view showing a portion of an example embodiment of a rod inserter;

FIG. 3D depicts a diagrammatic representation of a close-up view showing a portion of another example embodiment of a rod inserter;

FIG. 4 depicts a diagrammatic representation of a perspective view of an example bone screw;

FIG. 5A depicts a diagrammatic representation of a perspective view of an arrangement in which the rod shown in FIG. 1A is coupled to the rod inserter shown in FIGS. 3A and 3B, the rod being positioned in a collar of an example bone screw which is coupled to an example extender sleeve;

FIG. 5B depicts a diagrammatic representation of a side view of the arrangement shown in FIG. 5A;

FIG. 6 depicts a diagrammatic representation of a side view of an example embodiment of a counter-torque hand tool;

FIG. 7A depicts a diagrammatic representation of a side view of the arrangement shown in FIG. 5A without the rod inserter and with the counter-torque hand tool positioned over the extender sleeve;

FIG. 7B depicts a diagrammatic representation of another side view of the arrangement shown in FIG. 5A with the rod inserter and with the counter-torque hand tool positioned over the extender sleeve;

FIG. 7C depicts a diagrammatic representation of a top view of the arrangement shown in FIG. 7B, illustrating one embodiment of a method of shearing off a portion of the rod utilizing the rod inserter in conjunction with the counter-torque hand tool positioned over the extender sleeve;

FIG. 8 depicts a diagrammatic representation of a close-up view showing a portion of the rod of FIG. 1A that is broken off from the rest of the rod; and

FIG. 9 depicts a diagrammatic representation of a close-up view showing a portion of the rod of FIG. 1A after its removable end is broken off.

While this disclosure is susceptible to various modifications and alternative forms, specific embodiments thereof are shown by way of example in the drawings and will herein be described in detail. It should be understood, however, that the drawings and detailed description thereto are not intended to limit the disclosure to the particular form disclosed, but to the contrary, the intention is to cover all modifications, equivalents and alternatives falling within the spirit and scope of the present disclosure as defined by the appended claims.

### DETAILED DESCRIPTION

A surgical instrument for inserting and disengaging a rod ex situ and the various features and advantageous details thereof are explained more fully with reference to the non-limiting embodiments detailed in the following description. Descriptions of well known starting materials, manufacturing techniques, components and equipment are omitted so as not to unnecessarily obscure the invention in detail. Skilled artisans should understand, however, that the detailed description and the specific examples, while disclosing preferred embodiments of the invention, are given by way of illustration only and not by way of limitation. Various substitutions, modifications, and additions within the scope of the underlying inventive concept(s) will become apparent to those skilled in the art after reading this disclosure. Skilled artisans can also appreciate that the drawings disclosed herein are not necessarily drawn to scale.

As used herein, the terms “comprises,” “comprising,” includes, “including,” “has,” “having” or any other variation thereof, are intended to cover a non-exclusive inclusion. For example, a process, product, article, or apparatus that comprises a list of elements is not necessarily limited to only those elements, but may include other elements not expressly listed or inherent to such process, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Additionally, any examples or illustrations given herein are not to be regarded in any way as restrictions on, limits to, or express definitions of, any term or terms with which they are utilized. Instead, these examples or illustrations are to be regarded as being described with respect to a particular embodiment and as illustrative only. Those of ordinary skill in the art will appreciate that any term or terms with which these examples or illustrations are utilized encompass other embodiments as well as implementations and adaptations thereof which may or may not be given therewith or elsewhere in the specification and all such embodiments are intended to be included within the scope of that term or terms. Language designating such non-limiting examples and illustrations includes, but is not limited to: “for example,” “for instance,” “e.g.,” “in one embodiment,” and the like.

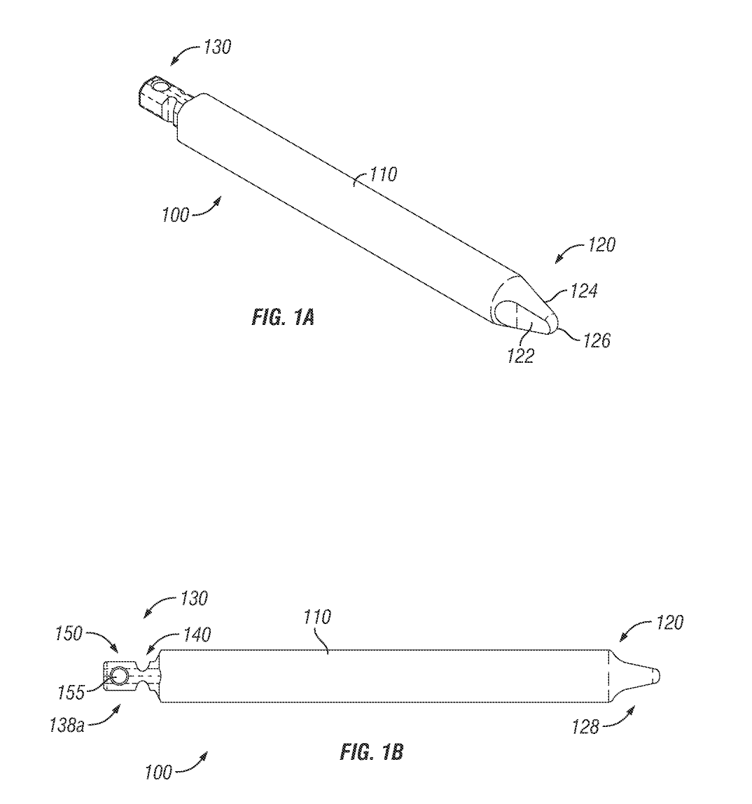

FIG. 1A depicts a diagrammatic representation of a perspective view of an example embodiment of a rod having a removable end. In this example, rod 100 comprises body 110, first end 120, and second end 130. Rod 100 may be made from various suitable biocompatible materials as known to those skilled in the art. Rod 100 may be straight or bent. Rod 100 or a portion thereof may be solid. In some embodiments, first end 120 is shaped, sized, and dimensioned or otherwise structured for insertion through an incision on a patient in a minimally invasive manner. First end 120 may be blunt or tapered to facilitate tissue dissection during minimally invasive insertion. As an example, in one embodiment, first end 120 comprises tip 126 and tapered surfaces 122, 124. To reduce trauma to the surrounding tissue during insertion of rod 100, in some embodiments, tip 126 and tapered surfaces 122, 124 may be machined to remove sharp edges or formed to have a rounded and smooth appearance. Other configurations of first end 120 are also possible.

FIG. 1B depicts a diagrammatic representation of a side view of the rod shown in FIG. 1A. Second end 130 is configured for tool engagement. In this example, second end 130 comprises part 150 that can be removed from the rod and breakable portion 140. Part 150 can, but does not have to, have a cylindrical or cubical body. For rotational control of rod 100, part 150 may have a cross-sectional profile of a triangle, hexagon, square, rectangle, and the like. Also, part 150 can, but does not have to, be circumferentially smaller than body 110. Those skilled in the art will appreciate that the shape, size, and configuration of part 150 may vary from implementation to implementation. In some embodiments, part 150, breakable portion 140, and body 110 are machined, formed, or otherwise constructed from a block of material. In some embodiments, part 150 is made from a material more rigid than breakable portion 140. In some embodiments, part 150 and breakable portion 140 are made from a material more rigid than that of body 110. In some embodiments, part 150 and breakable portion 140 are affixed to body 110 by adhesion. To ensure integrity of rod 100 near breakable portion 140, part 150 is preferably solid and not burred.

Differences between first end 120 and second end 130 of rod 100 can be ascertained by comparing profiles 128 and 138a. It can be seen from profile 138a that structural integrity of second end 130 of rod 100 is strategically weakened at breakable portion 140. In some embodiments, additional steps and/or features may be utilized to facilitate the removal of part 150. For example, breakable portion 140 may be scored. In some embodiments, materials having different properties, including physical and mechanical properties, may be utilized. For example, part 150 and body 110 may be made of materials having different tensile strengths and/or densities. Those skilled in the art will appreciate that breakable portion 140 can be implemented in many ways and is not limited by what is shown in FIG. 1B.

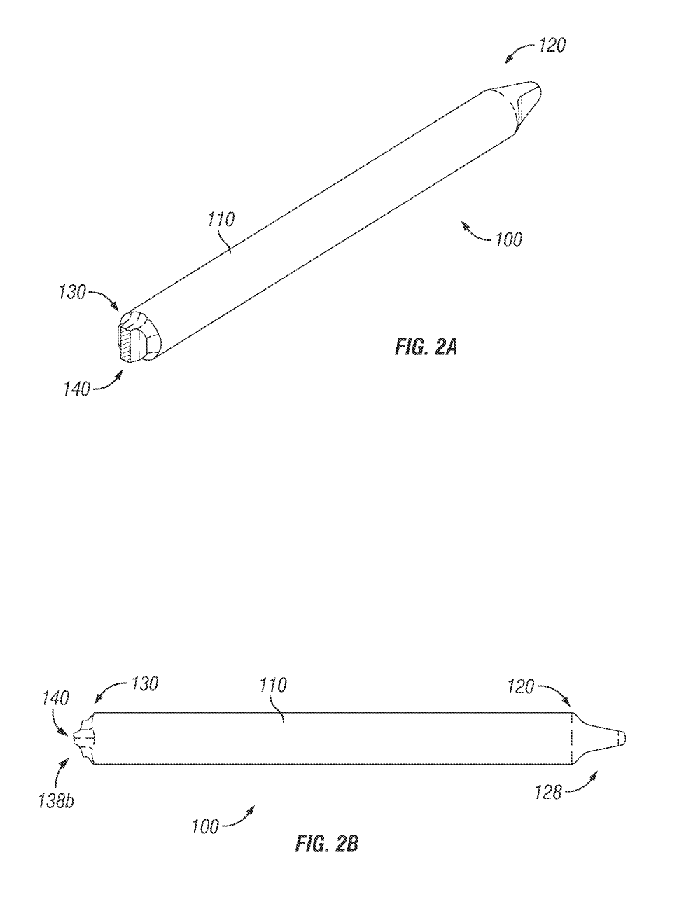

FIG. 2A depicts a diagrammatic representation of a perspective view of the rod shown in FIG. 1A after a portion of the rod is broken off or otherwise removed from the rod. In this example, part 150 is cut off or otherwise detached from second end 130 at breakable portion 140. In this embodiment, breakable portion 140 has a cross-sectional profile resembling a rectangle. FIG. 2B depicts a diagrammatic representation of a side view of the rod shown in FIG. 2A. Specifically, FIG. 2B shows profile 138b of second end 130 without part 150.

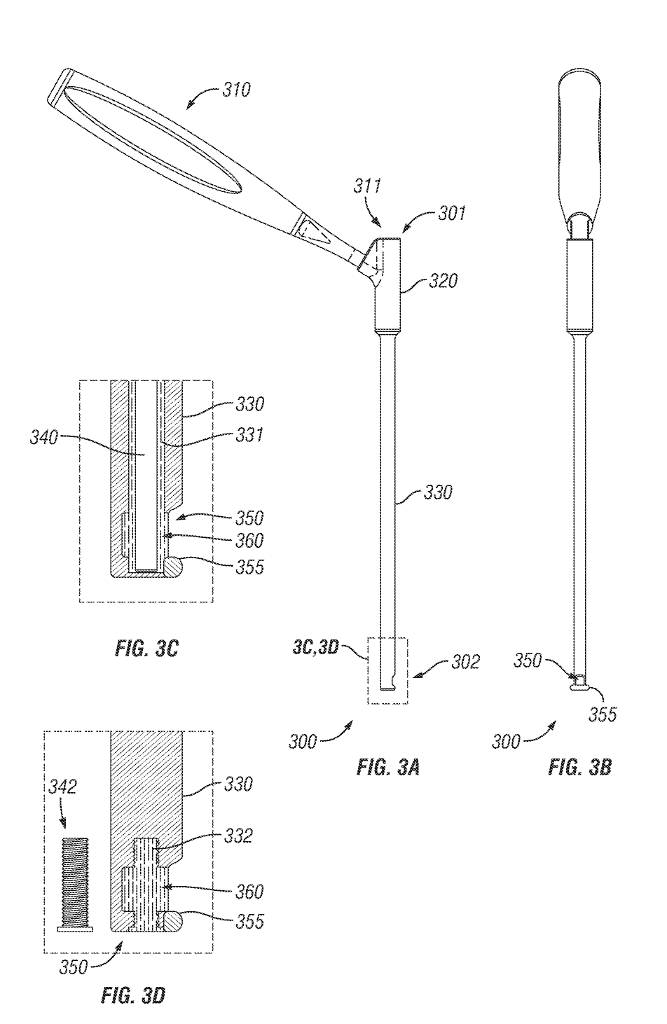

FIGS. 3A and 3B depict diagrammatic representations of side views of an example embodiment of a surgical tool for holding and inserting a rod and removing a portion of the rod after insertion. FIG. 3B is a diagrammatic representation of a second side view of the surgical tool shown in FIG. 3A. In this example, rod insertion tool or rod inserter 300 comprises handle 310, body 320, and shaft 330. Shaft 330 and body 320 may be centrally aligned while handle 310 may be positioned at an angle and offset from the central axis of body 320 and shaft 330.

In some embodiments, shaft 330 may be solid or hollow. FIG. 3C depicts a diagrammatic representation of a close-up view showing a portion of an example embodiment of a rod inserter with a hollow shaft. FIG. 3D depicts a diagrammatic representation of a close-up view showing a portion of another example embodiment of a rod inserter with a solid shaft.

Referring to FIG. 3C, shaft 330 of rod inserter 300 may have channel 331 extending substantially along the length of shaft 330. In this example, channel 331 is capped at distal end 302 of rod inserter 300. Referring to FIGS. 3A and 3C, locking pin 340 may be introduced into channel 331 of shaft 330 through opening 311 of body 320 at proximal end 301. In this case, opening 311 of body 320 is in communication with channel 331 of shaft 330. Referring to FIGS. 3A-3C, at distal end 302, shaft 330 may comprise rod holder 350. In some embodiments, distal end 302 of shaft 330 may comprise stop 355. Rod holder 350 may comprise cavity 360 that is shaped, sized, and dimensioned to receive part 150 of rod 100. In some embodiments, entrance to cavity 360 may be surfaced to allow part 150 to slide into rod holder 350.

Referring to FIG. 3D, shaft 330 of rod inserter 300 may be solid or substantially solid. In some embodiments, shaft 330 may be made of a solid piece of material. In some embodiments, shaft 330 may be machined or formed in conjunction with body 320 as a single piece. In some embodiments, shaft 330 and body 320 may be machined or formed separately and affixed to each other as a single piece. Referring to FIGS. 3A and 3D, rod 100 may comprise rod holder 350 at distal end 302. In some embodiments, rod holder 350 may comprise stop 355. In this example, distal end 302 has opening 332 that is female-threaded to mate with screw 342. In this embodiment, opening 332 extends from distal end 302, in communication with cavity 360 of rod holder 350. In some embodiments, screw 342 may be a set screw having a hex or slot drive recessed in the threaded length.

Referring to FIG. 1B, in some embodiments, second end 130 includes an insertion tool engagement feature. The engagement feature may be designed for mating with the insertion tool. For example, hole 155 of part 150 may be shaped, sized, and dimensioned to receive locking pin 340 or set screw 342. Having locking pin 340 or set screw 342 inside cavity 360 of rod holder 350 may prevent movement of part 150 relative to rod holder 350 and provide a surgeon with translational control of rod 100. In some embodiments, a method of inserting rod 100 utilizing rod insertion tool 300 may comprise inserting part 150 of rod 100 into cavity 360 of shaft 330 and inserting locking pin 340 into hole 155 of part 150 through opening 311 at proximal end 301 of body 320 and channel 331 of shaft 330. In some embodiments, the method may further comprising locking or fastening locking pin 340 onto hole 155, body 320, or both. For example, in some embodiments, a distal end portion of locking pin 340 may be male-threaded. In this case, hole 155 of part 150 may be female-threaded to mate with the threaded portion of locking pin 340. As another example, in some embodiments, a proximal end portion of locking pin 340 may be male-threaded. In this case, a portion of body 320 may be female-threaded in the interior to mate with the threaded portion of locking pin 340.

In some embodiments, a method of inserting rod 100 utilizing rod inserter 300 may comprise inserting part 150 of rod 100 into cavity 360 of shaft 330 and screwing or fastening set screw 342 through hole 155 of part 150 and into opening 332 of rod holder 350. In embodiments disclosed herein, rod 100 is attached to rod inserter 300 ex situ.

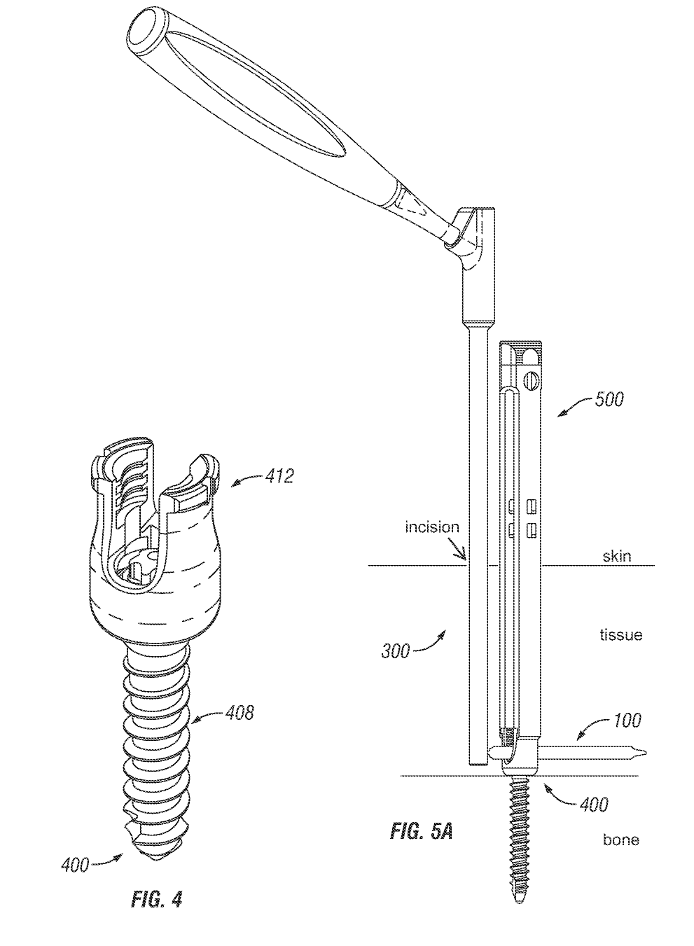

FIG. 4 depicts a diagrammatic representation of a perspective view of example bone screw 400 having collar 412 and shaft 408. Bone screw 400 may be utilized in a spinal stabilization system. Examples of a spinal stabilization procedure and surgical instruments used therein, including targeting needles, tissue dilators, rod pushers (seaters), universal drivers, screw adjusters, bone awls, bone taps, bone screws, and extender sleeves are described in co-pending U.S. patent application Ser. No. 11/284,282, filed Nov. 21, 2005, entitled “SPINAL STABILIZATION SYSTEMS AND METHODS,” which is a continuation of U.S. patent application Ser. No. 10/697,793, filed Oct. 30, 2003, issued as U.S. Pat. No. 7,250,052, entitled “SPINAL STABILIZATION SYSTEMS AND METHODS,” the contents of which are incorporated herein as if set forth in full. As used herein, the term “collar” includes any element that wholly or partially encloses or receives one or more other elements. A collar may enclose or receive elements including, but not limited to, a bone fastener, a closure member, a ring, and/or an elongated member. In some embodiments, a collar may couple two or more other elements together (e.g., an elongated member and a bone fastener). In some embodiments, a collar may have a “U” shape, however it is to be understood that a collar may also have other shapes.

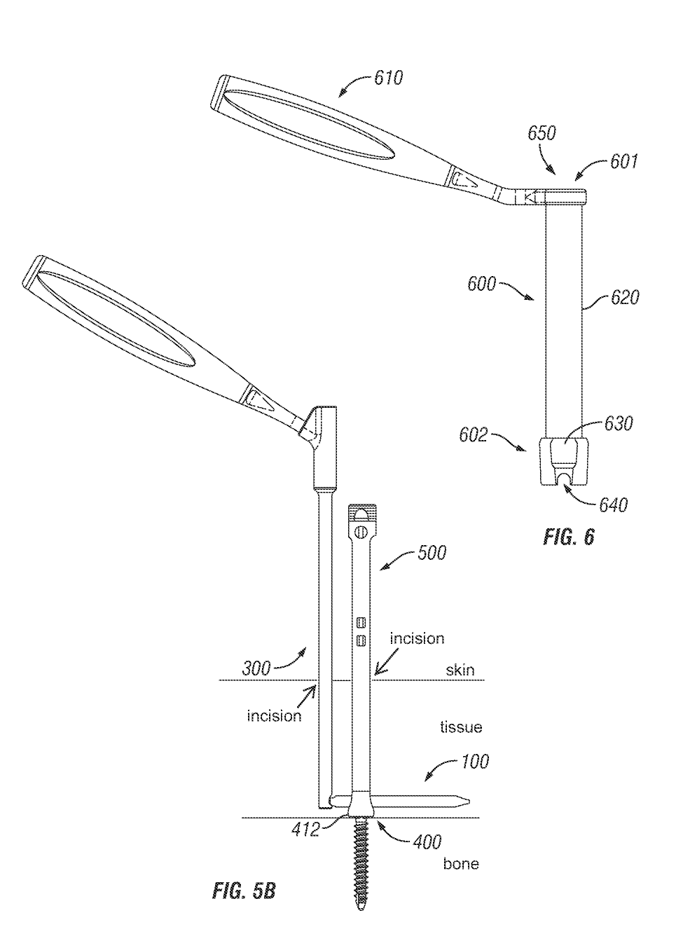

FIG. 5A depicts a diagrammatic representation of a perspective view of an arrangement in which rod 100 is coupled to rod inserter 300 ex situ, inserted into a surgical site percutaneously through a small incision, and positioned in collar 412 of bone screw 400. Bone screw 400 may be anchored in a vertebral body and coupled to extender sleeve 500. Examples of extender sleeves, bone fastener assemblies and ways to couple them to vertebral bodies are described in the above-referenced U.S. patent application Ser. No. 11/284,282 and U.S. Pat. No. 7,250,052. Extender sleeve 500 may be used as a guide to install bone screw 400 in a vertebra. Instruments may be inserted into extender sleeve 500 to manipulate bone screw 400. Movement of extender sleeve 500 may alter an orientation of collar 412 relative to bone screw 400.

FIG. 5B depicts a diagrammatic representation of a side view of the arrangement shown in FIG. 5A. After rod 100 is properly positioned (seated) in collar 412, a surgeon may secure rod 100 to bone screw 400 with a closure member. As disclosed in the above-referenced U.S. patent application Ser. No. 11/284,282 and U.S. Pat. No. 7,250,052, a driver may be used to position and lock the closure member in collar 412 of bone screw 400. The driver may include a handle, a shaft, and a coupling portion for engaging the tool portion of the closure member through the hollow interior of extender sleeve 500. The closure member may couple to collar 412 by a variety of systems including, but not limited to, standard threads, modified threads, reverse angle threads, buttress threads, or helical flanges. The closure member may be advanced into an opening in collar 412 to engage a portion of rod 100 and inhibit movement of rod 100 relative to collar 412. A bottom surface of the closure member may include structure and/or texturing to enhance the ability of the closure member to secure rod 100 in collar 412. After rod 100 is secured in collar 412, the surgeon may apply counter torque to extender sleeve 500 above the incision while the tool portion of the closure member is broken off using the driver.

FIG. 6 depicts a diagrammatic representation of a side view of an example embodiment of a counter-torque hand tool. In this example, counter-torque hand tool 600 may comprise handle 610 and body 620. Body 620 may comprise proximal end 601, distal end 602, and hollow interior or channel 650 extending from proximal end 601 to distal end 602. Channel 650 may be sized and dimensioned to slide over extender sleeve 500. In some embodiments, channel 650 may be structured to complement the exterior of extender sleeve 500. Distal end 602 of body 620 may comprise notches 640 for coupling counter-torque hand tool 600 to rod 100. Notches 640 may be of sufficient size and dimension to complement the shape and size of rod 100. In this example, “U” shaped notches 640 are symmetrically positioned at the rim of distal end 602 of counter-torque hand tool 600.

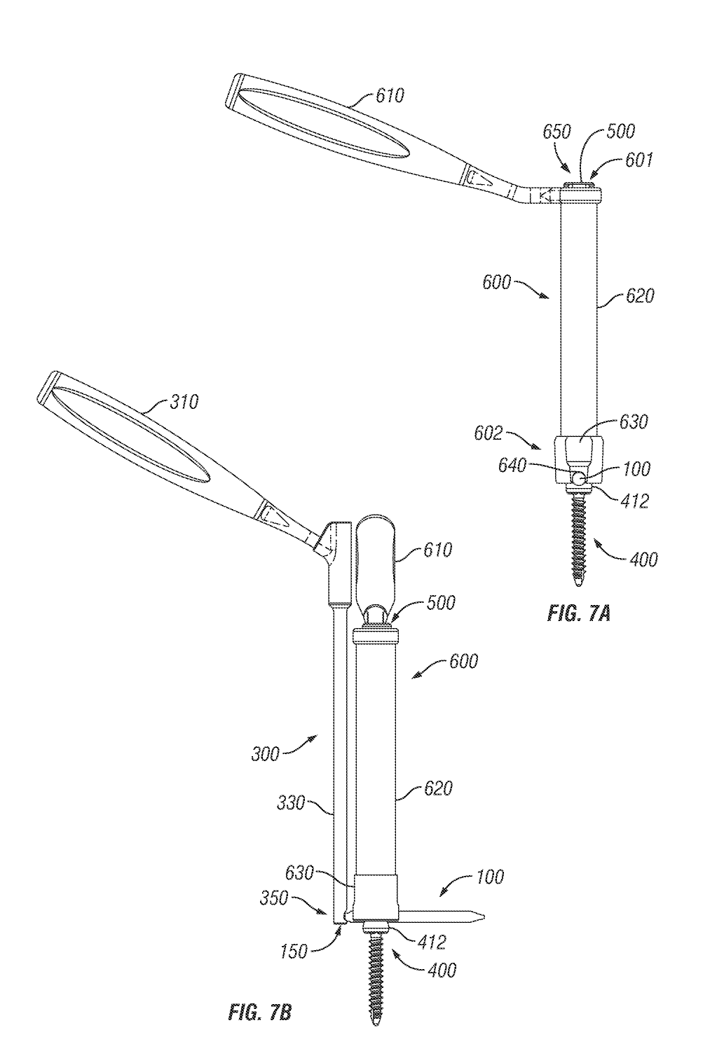

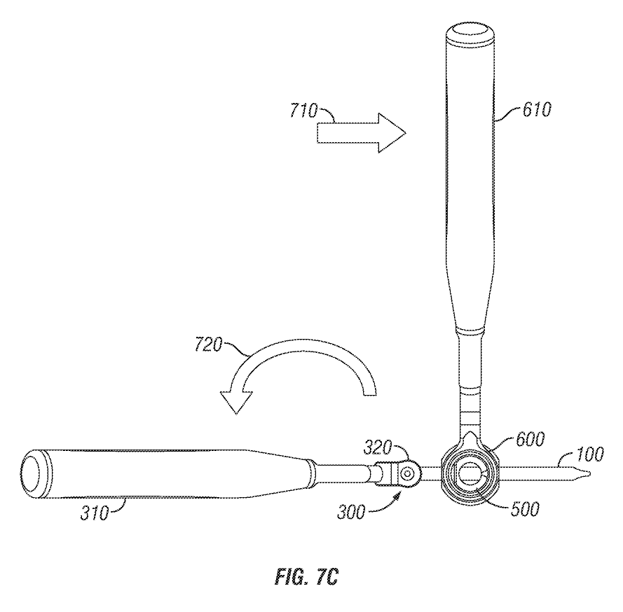

FIG. 7A depicts a diagrammatic representation of a side view showing counter-torque hand tool 600 positioned over extender sleeve 500 through channel 650, with distal end 602 of body 620 coupled to rod 100 via notches 640. Extender sleeve 500 is coupled to collar 412 of bone screw 400 as described above. Distal end 602 of body 620 may further comprise flat or substantially flat surface 630. Surface 630 may allow a minimal space between counter-torque hand tool 600 and rod inserter 300.

FIG. 7B depicts a diagrammatic representation of another side view of showing rod holder 350 of rod inserter 300 securely holding part 150 of rod 100, with counter-torque hand tool 600 positioned over extender sleeve 500 coupled to collar 412 of bone screw 400 which securely holds body 110 of rod 100. As FIGS. 7A-7B illustrate, a surgeon may fixedly engage counter-torque hand tool 600 with rod 100 over extender sleeve 500. Rod 100, in turn, is locked onto bone screw 400 which is anchored in a vertebral body in a manner known to those skilled in the art.

FIG. 7C depicts a diagrammatic representation of a top view of the arrangement shown in FIG. 7B, illustrating one embodiment of a method of removing a portion of the rod utilizing the rod inserter in conjunction with the counter-torque hand tool positioned over the extender sleeve. More specifically, to shear off part 150 of rod 100 securely held by rod holder 350 of rod inserter 300, a surgeon may turn or twist handle 310 of rod inserter 300 in the direction of arrow 720 while holding onto handle 610 of counter-torque hand tool 600 and applying counter torque in the direction of arrow 710. Part 150 may snap off with one turn. For example, part 150 may sever or break off from rod 100 at breakable portion 140 with a 90° or approximately 90° turn of handle 310 of rod inserter 300. In some cases, it may take a few back-and-forth motions to separate part 150 from rod 100. In some embodiments, to facilitate a clean breakage, stop 355 may be strategically sized and/or placed near breakable portion 140.

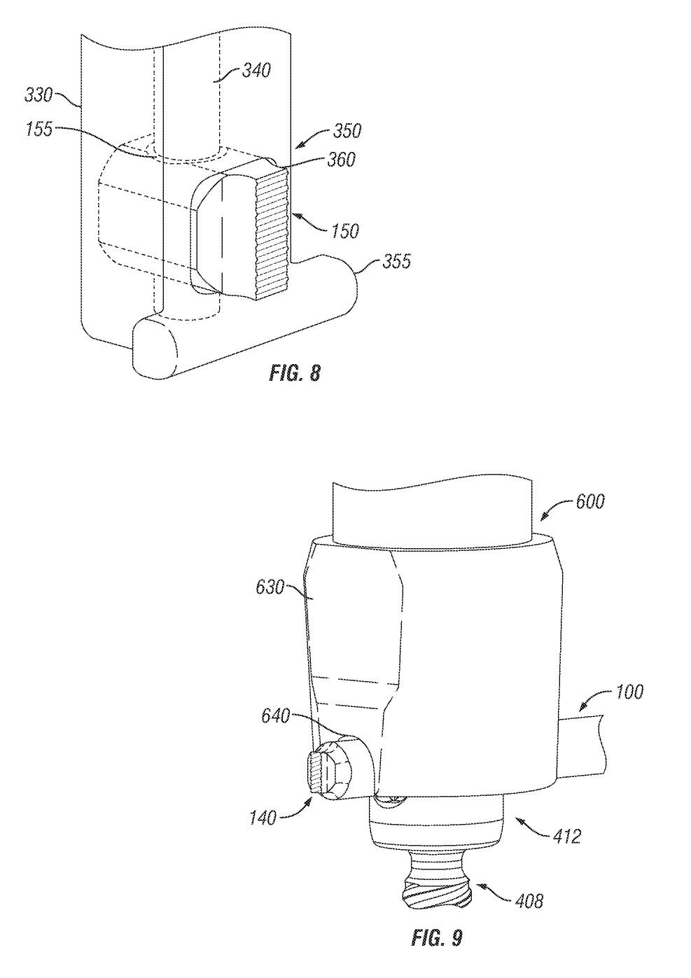

FIG. 8 depicts a diagrammatic representation of a close-up view showing part 150 of rod 100 held inside cavity 360 of rod holder 350 via locking pin 340 protruding through hole 155, after part 150 is broken off or otherwise separated from the rest of rod 100. Once part 150 is separated from rod 100, rod inserter 300—with part 150 securely held inside cavity 360 of rod holder 350—can be retrieved from the surgical site and part 150 can be unlocked and removed from rod holder 350 ex situ. In some embodiments, unlocking part 150 from rod holder 350 may comprise pulling and/or loosening locking pin 340. In some embodiments, unlocking part 150 from rod holder 350 may comprise loosening set screw 342.

FIG. 9 depicts a diagrammatic representation of a close-up view showing a portion of rod 100 after part 150 is broken off or otherwise separated. Counter-torque hand tool 600 can now be removed, leaving extender sleeve 500 coupled to collar 412 of bone screw 400. Depending upon the number of rods needed, the above-described percutaneous rod insertion may be performed one or more times in a minimally invasive surgical procedure, each time inserting an embodiment of the rod disclosed herein utilizing an embodiment of the rod inserter disclosed herein, shearing off an end of the rod in situ, and removing the broken off end of the rod from the rod inserter ex situ.

Embodiments of a rod with a removable end and a minimally invasive rod inserter have now been described in detail. Modifications and alternative embodiments of various aspects of the disclosure will be apparent to those skilled in the art in view of this description. Accordingly, this description is to be construed as illustrative only and is for the purpose of teaching those skilled in the art the general manner of carrying out the disclosure. It is to be understood that the forms of the disclosure shown and described herein are to be taken as examples of embodiments. Elements and materials may be substituted for or implemented from those illustrated and described herein, as would be apparent to one skilled in the art after having the benefit of the disclosure. Changes may be made in the elements or to the features described herein without departing from the spirit and scope of the disclosure as set forth in the following claims and their legal equivalents.

What is claimed is:

1. A spinal stabilization rod suitable for minimally invasive insertion, comprising:
an elongated body;
a first end that is configured to facilitate tissue dissection; and
a second end that is opposite of the first end and having a part and a breakable portion located between the part and the elongated body, the breakable portion having a size smaller than the part and the elongated body.

2. The spinal stabilization rod of claim 1, wherein the part comprises a non-circular cross-sectional profile.

3. The spinal stabilization rod of claim 1, wherein the part comprises a through hole.

4. The spinal stabilization rod of claim 1, wherein the elongated body, the breakable portion, and the part are made of same material.

5. The spinal stabilization rod of claim 1, wherein the elongated body and the part are made of different materials.

6. The spinal stabilization rod of claim 5, wherein the part is more rigid than a transition between the part and the elongated body.

7. The spinal stabilization rod of claim 5, wherein the part is affixed to the elongated body at the breakable portion.

8. The spinal stabilization rod of claim 1, wherein the elongated body is straight or bent.

9. The spinal stabilization rod of claim 1, wherein the breakable portion has a non-circular cross-sectional profile.

10. An apparatus for minimally invasive rod insertion, comprising:
a handle; and
a body connected to the handle and having:
a proximal end;
a distal end;
a shaft between the proximal end and the distal end; and
a rod holder at the distal end,
wherein the rod holder comprises:
a cavity, wherein the cavity is sized and dimensioned to accommodate a part of a rod; and
an opening in communication with the cavity, wherein the opening is structured to receive a pin or a screw for locking the part of the rod in the cavity of the rod holder.

11. The apparatus of claim 10, wherein the opening extends from the proximal end of the body and stops at the distal end of the body, beyond the cavity of the rod holder.

12. The apparatus of claim 11, wherein at least a portion of the opening is female-threaded to mate with at least a portion of the pin.

13. The apparatus of claim 10, wherein the shaft is solid or substantially solid.

14. The apparatus of claim 13, wherein the opening extends from the distal end of the body and is female-threaded to mate with the screw.

15. The apparatus of claim 14, wherein the screw is a set screw.

16. The apparatus of claim 10, wherein the rod holder further comprises:
a stop formed at entrance to the cavity.

17. A method for minimally insvasive insertion of a spinal stabilization rod, comprising:
coupling a part of a rod to a rod holder of a rod inserter;
securing the part of the rod to the rod holder to prevent movement of the part of the rod relative to the rod holder of the rod inserter;
inserting the rod through an incision;
positioning the rod in a collar of a bone screw anchored in a vertebral body;
coupling an extender sleeve to the collar of the bone screw to prevent movement of the rod relative to the bone screw;
coupling a tool over the extender sleeve and a portion of the collar of the bone screw and onto the rod;
holding the rod in place via the tool and manipulating the rod inserter to remove the part of the rod in situ and release the rod inserter from the rod; and
removing the rod inserter from the incision, with the part of the rod coupled to the rod holder of the rod inserter.

18. The method according to claim 17, wherein the part of the rod is securely held by a pin or screw inside the rod holder of the rod inserter.

19. The method according to claim 18, further comprising:
unlocking the pin or screw to remove the part of the rod from the rod holder of the rod inserter ex situ.

20. The method according to claim 17, wherein holding the rod in place via the tool and manipulating the rod inserter cause the rod to break at a structurally weakened point between the part and the rod.

21. The method according to claim 17, wherein the part of the rod comprises a non-circular cross-sectional profile.

22. The method according to claim 17, wherein the opening of the rod inserter extends from a proximal end, through a hollow shaft, and stops at a distal end of the rod inserter, beyond a cavity of the rod holder.

23. The method according to claim 17, wherein the opening of the rod inserter extends from a distal end towards a proximal end of the rod inserter and is female-threaded to mate with the screw.

24. The method according to claim 17, wherein the rod comprises a blunt or tapered end to facilitate tissue dissection.

25. The method according to claim 17, wherein the tool is coupled to the rod via symmetrically positioned notches at a distal end of the tool.

26. The method according to claim 17, wherein the tool is a counter-torque hand tool.
